(19) United States
(12) Patent Application Publication
(10) Pub. No.: US 2026/0224479 A1
(43) Pub. Date: Aug. 6, 2026

(54) ORAL PRODUCT FOR DELIVERY OF AN ACTIVE INGREDIENT

(71) Applicant: CONTRAF-NICOTEX-TOBACCO GmbH, Heilbronn (DE)

(72) Inventors: Thomas ELLERICHMANN, Heilbronn (DE); Markus BELZ, Heilbronn (DE); Thorsten KÖNIG, Heilbronn (DE)

(21) Appl. No.: 19/152,850

(22) PCT Filed: Jan. 23, 2024
(86) PCT No.: PCT/EP2024/051466

§ 371 Date: Jul. 31, 2025

(30) Foreign Application Priority Data
Feb. 3, 2023 (EP) 23154874.4

(51) Int. Cl. A61K 9/00; A61K 9/06; A61K 31/00; A61K 31/4045; A61K 31/465; A61K 31/522; A61K 47/02; A61K 47/10; A61K 47/26; A61K 47/36; A61K 47/38

(52) U.S. Cl. CPC A61K 9/0056; A61K 9/06; A61K 31/4045; A61K 31/465; A61K 31/522; A61K 31/658; A61K 47/02; A61K 47/10; A61K 47/26; A61K 47/36; A61K 47/38

(57) ABSTRACT

The invention relates to an oral product for delivery of an active ingredient comprising or consisting of at least one layer and a composition comprising a hydrogel forming component and the active ingredient. The oral product is capable of releasing the active ingredient without the need of chewing the oral product.

### FIELD OF THE INVENTION

The present invention relates to an oral product for delivery of an active ingredient.

### BACKGROUND OF THE INVENTION

Tobacco smoking is the world's leading cause of avoidable premature deaths, reflecting the high toxicity of tobacco smoke inhaled by smokers over a long time. The harm done by tobacco smoke, especially its influence on the occurrence of lung cancer, is known for decades. Nevertheless, there are more than 1.2 billion tobacco smokers worldwide. However, nicotine as an active ingredient may have some positive effects to the consumer for example reduction of body weight, enhancement of physical performance and protection against diseases, especially against Parkinson's disease, Tourette's disease, Alzheimer's disease and sleep apnoea.

Therefore, some alternatives to tobacco smoking for consumption of nicotine became widely available, for example electronic cigarettes, Heat-not-Burn (HnB) devices or oral nicotine products with or without tobacco.

Electronic cigarettes and Heat-not-Burn (HnB) devices show some progress regarding the health risks involved with smoking, but also have certain disadvantages, as aerosols of varying compositions are still inhaled. Especially in the case of HnB devices potentially harmful substances are directly administered into the lungs.

Oral nicotine products with or without tobacco have the advantage of avoiding inhalation of harmful substances. For example, tobacco containing pouches as oral products (so called Swedish Snus) first introduced in 1973, following the Gothiatek Standard introduced by Swedish Match in the early 2000s, show great harm reduction potential in Sweden and other Scandinavian countries. The lung cancer rates in Sweden as well as the smoking rates among the population are today among the lowest in the world. The Gothiatek Standard is a quality system that ensures (among other parameters) low contents of harmful substances such as heavy metals and tobacco specific nitrosamines (TSNAs) in the products. However, tobacco containing pouches still comprise these harmful substances, albeit in small quantities.

Therefore, in the upcoming years there were several attempts to develop tobacco free pouches containing nicotine as oral products. The first commercial approach was the Zonnic brand, introduced by Niconovum in 2008, as smoking cessation aid. It took several years to develop this new category into a consumer product. However, the tobacco free oral products are a promising way of nicotine consumption with even more potential harm reduction, as there are no heavy metals or TSNAs to be expected, if the ingredients including nicotine are of adequate quality and composition.

Despite the progress already made in the development of tobacco free oral products containing active ingredients, in particular nicotine, there remains further need to improve the replacement of a habit like tobacco smoking, with an oral product that is close to smoking in the categories of user experience, in particular easy to be used, the price, sensory attractiveness and a fast relief of a craving.

The present invention addresses all of these points, as the inventive oral product allows a comparatively faster release of nicotine and subsequent faster uptake of nicotine, a superior sensory experience and an easy and low effort to produce the oral product. Furthermore, as another advantage of the oral product the nicotine can be replaced or combined with any other active ingredients, in particular caffeine.

### Object and Solution

In view of the foregoing, the object underlying the present invention is therefore to make available an oral product which properly addresses the above-mentioned need.

This object is accomplished by an oral product according to independent claim 1. Preferred embodiments of the oral product are defined in the dependent claims 2 to 15. Further preferred embodiments of the invention are defined in the present description. The subject-matter and wording, respectively of all claims is hereby incorporated into the description by explicit reference.

According to a first aspect, the present invention relates to an oral product, in particular for delivery of an active ingredient, comprising at least one layer and a composition comprising a hydrogel forming component and the active ingredient. The oral product, preferably due to the water-soluble nature of the composition, is capable of releasing the active ingredient without the need of chewing the oral product.

Preferably, the oral product, in particular the composition, is capable of releasing the active ingredient by contact with mucosa, saliva, water and/or aqueous solutions.

Preferably, the oral product, in particular for delivery of an active ingredient, consists of at least one layer and a composition comprising a hydrogel forming component and the active ingredient.

In other words, the active ingredient is contained in the composition.

The oral product or at least the at least one layer may be at least partly, in particular only partly or preferably completely, biodegradable.

An advantage of a biodegradable oral product is a reduced risk of environmental pollution from the oral product, as it allows for rapid degradation in the environment and a reduction of plastic waste due to biodegradable layers.

Preferably, the oral product is wholly receivable in an oral cavity, in particular an oral cavity between gum and cheek.

Further preferred, the composition is a water-soluble composition.

The term “oral product” as used according to the present invention means a product which is intended to be taken into the mouth and preferably to be in contact with the oral mucosa while it remains in the mouth for a period of time but is not chewed or swallowed.

The term “layer” as used according to the present invention means a layer of a material, in particular a layer of a fabric material, which includes various fiber-based materials, including but not limited to fibers, yarns, filaments and threads.

The term “at least one layer” as used according to the present invention means one layer or a plurality of layers, i.e. two or more layers.

The term “biodegradable”, as used according to the present invention, means a at least partial degradation or decomposition, i.e. chemical breakdown, of a material when being exposed to environmental influence, in particular rain, humidity and sunray.

The term “active ingredient” as used according to the present invention means a substance which in an organism has a specific action and/or evokes a specific response.

The term “pouch” as used according to the present invention means a closed container or bag, providing a room for storage in its inside.

The present invention is characterized in particular by the following advantages:
The oral product allows a comparatively faster release of active ingredients, in particular nicotine, and subsequent a faster uptake of the active ingredient.
The product shows a high overall stability.
A superior sensory experience can be achieved by the oral product.
Manufacturing effort and production costs are low due to the layer structure.
The oral product is suitable for the delivery of multiple active ingredients other than nicotine.
The consumer dosage options of the active ingredient are better through the variable dimensions of the oral product.
The oral product allows a comparatively faster release of additive compounds, in particular flavours.
A chewing action is not needed for the release of the active ingredient, thereby a more sustained and consistent release of the active ingredient can be achieved.

In an embodiment of the invention the at least one layer is coated or layered by the composition.

In other words, it can be preferable according to the invention for the composition to be in the form of at least one layer of coating or layering on the at least one layer.

This is in particularly advantageous since the layer coated or layered by the composition and the composition layer itself are wetted in the mouth at the same time due to contact with the saliva. Subsequently the composition, in particular water-soluble composition, preferably dissolves and a fast release of the active ingredient and a subsequent fast uptake through the oral mucosa can be achieved. A need for a chewing action to release the active ingredient from the oral product, which may lead to an unbalanced release of the active ingredient, is not given.

A further advantage of the at least one layer being coated or layered by the composition is, that the oral product allows a faster and more consistent release of the active ingredient, in particular nicotine, and a subsequent faster and more consistent uptake of the active ingredient, compared to known oral products, in particular compared to oral products that are in the need of chewing to release ingredients, in particular active ingredients.

Preferably, the composition layer may have a layer thickness of 0.1 mm to 2.5 mm, in particular of 0.5 mm to 1.5 mm, preferably of 0.9 mm to 1.1 mm.

Further, the composition may have a proportion of 0.001% by weight to 80% by weight, in particular 1% by weight to 70% by weight, preferably 10% by weight to 60% by weight, more preferably 50% by weight to 60% by weight, based on the total weight of the oral product.

In a further embodiment of the invention the composition penetrates the at least one layer. In other word the at least one layer is penetrated by the composition.

Alternatively, the at least one layer is coated or layered by the composition and in addition the composition penetrates the at least one layer.

Preferably, when penetrating the at least one layer, the composition forms an inner composition layer within the at least one layer.

In other words, it can be preferable according to the invention for the composition to be in the form of an inner layer within the at least one layer.

This is in particularly advantageous since the layer penetrated by the composition is wetted in the mouth due to contact with the saliva. Only after the layer is wetted the inner composition layer, in particular water-soluble composition layer, is wetted and preferably dissolves and a consistent fast release of the active ingredient from the layer and uptake through the oral mucosa and/or the gastro-intestinal system can be achieved. Therefore, there is no need for a chewing action to release the active ingredient from the oral product, which may lead to an unbalanced release of the active ingredient.

A further advantage of the at least one layer being penetrated by the composition is, that the oral product allows a faster and more consistent release of the active ingredient, in particular nicotine, and a subsequent faster and more consistent body uptake of the active ingredient, compared to known oral products, in particular compared to oral products that are in the need of chewing to release ingredients, in particular active ingredients.

In a further embodiment of the invention the at least one layer is in the form of only one layer.

In other words, it can be preferable according to the invention for the oral product to be a single layer oral product, in particular comprising the inventive composition.

Alternatively, the oral product may be in the form of a plurality of layers, in particular 2 to 5 layers, wherein the layers are arranged one above the other.

In other words, it can be preferable according to the invention for the oral product to be a multi-layer oral product, in particular comprising the inventive composition.

Preferably, the at least one layer is in the form of a plurality of layers, in particular 3 layers, wherein the layers are arranged one above the other.

In a further embodiment of the invention at least one inner layer, in particular only one inner layer, of the plurality of layers is coated (layered) and/or penetrated by the composition.

Alternatively, at least one outer layer, in particular only one outer layer or two opposing outer layers of the plurality of layers may be coated (layered) and/or penetrated by the composition.

Further, the composition may coat (layer) and/or penetrate the middle layer in case of three layers being arranged one above the other.

Preferably, one layer is located above the inner layer, coated (layered) and/or penetrated by the composition, and one layer is located underneath the inner layer, coated and/or penetrated by the composition.

In a further embodiment of the invention the layers are connected, in particular mechanically connected, to each other, preferably by means of stitching, embossing (imprinting) or needle felting.

The term “needle felting” as used according to the present invention means a mechanical connection of layers which is achieved when a needle is stabbed into layers, preferably arranged one above the other, multiple times. This process helps to entangle the material of the layers on itself leading to layers which are sticking together. The layers become firmly entangled together the more the needle is stabbed into it making the connection denser and firmer.

The term “stitching”, also known as sewing, as used according to the present invention means a mechanical connection of layers, which is achieved when a needle connected with at least one thread is used to stab into layers, preferably arranged one above the other, multiple times, while the layers are connected by the help of the at least one thread.

The term “embossing” or “imprinting” as used according to the present invention means a mechanical connection of layers which is achieved when a stamp is put onto layers, preferably arranged one above the other, at least one time, preferably with a certain force and/or heat, to connect the layers.

The mechanical connection of the layers ensures that the inventive oral product comprising or consisting of at least two layers, in particular at least two fabric layers, stays intact and does not dissolve and/or separate during use.

Due to a mechanical connection of the layers by stitching, embossing or needle felting the manufacturing effort and production costs are relatively low, compared to other means of mechanically connecting layers, in particular by using chemical adhesives.

In a further embodiment of the invention the at least one layer is in the form of at least one textile layer or in the form of at least one non-textile layer.

Preferably the at least one layer, in particular at least one textile layer, is in the form of at least one nonwoven fabric, at least one felt fabric, at least one knitted fabric, at least one woven fabric or combinations of at least two of the afore-said fabrics.

Preferably, the at least one layer is in the form of at least one nonwoven fabric.

In a further embodiment of the invention the at least one layer, in particular at least one textile layer, comprises or consists of water-insoluble fibres, in particular water-insoluble natural fibres, preferably water-insoluble plant fibres and/or water-insoluble animal fibres, and/or synthetic fibres or combinations of at least two of the afore-said fibres.

The natural fibres are preferably selected from the group consisting of cotton fabric, silk fabric, linen fabric, wool fabric, paper fabric and combinations of at least two of the afore-said natural fibres.

The synthetic fibres are preferably selected from the group consisting of lycra fabric, viscose fabric, nylon fabric, polyester fabric, rayon fabric, polyethylenterephthalat (PET) fabric and combinations of at least two of the afore-said synthetic fibres.

The non-textile layer is preferably selected from the group consisting of plastic layers, latex layers, foam layers rubber layers and combinations of at least two of the afore-said non-textile layers.

The at least one layer may have a proportion of 20% by weight to 99.999% by weight, in particular 25% by weight to 50% by weight, preferably 25% by weight to 35% by weight, based on the total weight of the oral product.

The at least one layer may have a thickness of 0.1 mm to 5 mm, in particular of 0.75 mm to 2 mm, preferably of 0.9 mm to 1.1 mm.

The afore-mentioned thickness of the at least one layer is especially advantageous for a sustained and consistent release of the active ingredient, in particular nicotine.

Preferably, the at least one layer and the layer of composition may be equal or different in terms of layer thickness.

The oral product comprising or consisting of the at least one layer and the composition comprising a hydrogel forming component and the active ingredient may have a thickness of 0.6 mm to 15 mm, in particular of 1 mm to 8 mm, preferably of 2 mm to 5 mm.

In a further embodiment of the invention the oral product, in particular the composition, is free of water.

Preferably, the oral product, in particular the composition, is substantially free of water.

The term “substantially free of water” as used according to the present invention means less than 10% by weight, in particular less than 1% to 5% by weight, water.

Preferably, the oral product, in particular the composition, may be free of water, in particular substantially free of water, after the oral product, in particular the composition, is dried.

In a further embodiment of the invention the composition is in the form of a hydrogel.

Preferably, the composition is in the form of a hydrogel before the oral product, in particular the composition, is dried.

The term “hydrogel” as used according to the present invention means a water containing polymer network which comprises a hydrogel forming component, preferably a long chain polysaccharide or protein.

In a further embodiment of the invention the hydrogel forming component may be selected from the group consisting of carrageen, in particular kappa carrageen, agar-agar, alginates, polyvinyl pyrrolidone, gelatine, polyvinyl acetate, cellulose derivates, in particular carboxy methylcellulose (CMC) or hydroxypropyl methylcellulose (HPMC), and mixtures of at least two of the afore-said hydrogel forming components.

In a further embodiment of the invention the active ingredient is in a free form, i.e. not ionic and/or bound form, in an ionic form, i.e. in the form of a salt, or in a bound, in particular complexed, form.

The active ingredient may be bound to a carrier, in particular a polymer, preferably selected from the group consisting of mineralic polymers, natural polymers, synthetic polymers and mixtures of at least two of the afore-said carriers.

Alternatively, the active ingredient may be bound to an ion-exchange resin, in particular selected from the group consisting of copolymers of divinylbenzene and methacrylic acid and mixtures of at least two of the afore-said ion-exchange resins.

Alternatively, the active ingredient may form a complex, in particular with cyclodextrins.

In a further embodiment of the invention the active ingredient is selected from the group consisting of alkaloids, in particular nicotine, anatabine, caffeine, theobromine or theophylline, natural or synthetic cannabinoids, in particular cannabinol, cannabidivarin, cannabichromen, cannabidiol, cannabigerol, tetrahydrocannabivarin, Delta-8-tetrahydrocannabinol, Delta-9-tetrahydrocannabinol or Delta-x-tetrahydro cannabidiol, hormones, in particular melatonin, amino acids, in particular taurine or lysine, vitamins, minerals and mixtures of at least two of the afore-said active ingredients.

Preferably, the active ingredient is nicotine and/or caffeine.

Advantageously, the composition enables the oral product for the delivery of multiple active ingredients other than nicotine.

Further, the oral product and/or composition may be free of tobacco or may contain or comprise tobacco.

In a further embodiment of the invention the active ingredient has a proportion of 0.001% by weight to 50% by weight, in particular 0.5% by weight to 30% by weight, preferably 1% by weight to 10% by weight, based on the total weight of the composition.

The afore-mentioned quantities of the active ingredient are especially advantageous for a sustained release of the active ingredient, in particular of nicotine.

In a further embodiment of the invention the active ingredient is released from the composition or oral product during a period of 0.5 min to 60 min, in particular 1 min to 30 min, preferably 1 min to 10 min.

In a further embodiment of the invention the oral product, in particular the composition, comprises at least one additive compound, in particular one or more additive compounds, preferably selected from the group consisting of salts, in particular sodium chloride, pH regulator, flavour, sugar substitute, sweetener, in particular high intensity sweetener, humectant, colour, preservative, and mixtures of at least two of the afore-said additive compounds.

Alternatively, the oral product, in particular the composition, comprises no additive compounds, in particular none of the afore-mentioned additive compounds.

A further advantage of the at least one layer being coated (layered) and/or penetrated by the composition comprising at least one additive compound is, that the oral product allows a faster and more consistent release of the additive compound, in particular of flavours, compared to known oral products, in particular compared to oral products that are in the need of chewing to release ingredients, in particular additives.

The hydrogel forming component may have a proportion of 0.01% by weight to 50% by weight, in particular 0.1% by weight to 10% by weight, preferably 0.1% by weight to 3% by weight, based on the total weight of the composition.

The pH regulator may be selected from the group consisting of carbonates, in particular sodium carbonate (Na₂CO₃), hydroxides, in particular sodium hydroxide (NaOH), organic acids, in particular citric acid, and salts thereof, fatty acids and salts thereof and mixtures of at least two of the afore-said pH regulators.

The pH regulator may have a proportion of 0.1% by weight to 10% by weight, in particular 0.5% by weight to 5% by weight, preferably 1% by weight to 3% by weight, based on the total weight of the composition.

The composition may have a pH of 4 to 9, in particular a pH of 7 to 9, preferably a pH of 7.5 to 8.5.

The flavour may have a proportion of 0.01% by weight to 50% by weight, in particular 0.1% by weight to 10% by weight, preferably 1% by weight to 5% by weight, based on the total weight of the composition.

The sugar substitute may be selected from the group consisting of polyols, in particular erythritol, xylitol, mannitol, maltitol, maltodextrin and mixtures of at least two of the afore-said polyols.

The sugar substitute may have a proportion of 0.01 % by weight to 50% by weight, in particular 0.1% by weight to 10% by weight, preferably 1% by weight to 5% by weight, based on the total weight of the composition.

The sweetener, in particular high intensity sweetener, may be selected from the group consisting of sucralose, aspartame, stevia extract, acesulfame-K and mixtures of at least two of the afore-said sweeteners.

The sweetener, in particular high intensity sweetener, may have a proportion of 0.01% by weight to 10% by weight, in particular 0.1% by weight to 10% by weight, preferably 1% by weight to 5% by weight, based on the total weight of the composition.

The humectant may have a proportion of 0.01% by weight to 50% by weight, in particular 0.1% by weight to 10% by weight, preferably 1% by weight to 5% by weight, based on the total weight of the composition.

The composition may form a hydrogel with a proportion of water of 10% by weight to 98% by weight, in particular 50% by weight to 75% by weight, preferably 65% by weight to 75% by weight, based on the total weight of the formed hydrogel.

The colour may be selected from the group consisting of approved food colours and mixtures of at least two approved food colours.

The preservative may be selected from the group consisting of approved food preservatives and mixtures of at least two approved food preservatives.

The afore-mentioned quantities of the additives are especially advantageous for a sustained and consistent release of the additive compounds in particular of the additive compounds functioning as technical aids, for example pH regulators, and the additive compounds functioning as sensorial aids, for example flavours and sweeteners, from the oral product, in particular from the composition.

Advantageously, the composition preferably enables the oral product for the delivery of multiple additive compounds.

In a further embodiment of the invention the oral product is in the form or shape of a pouch, sucking article such as bonbon, or a lozenge.

Advantageously, the oral product is easily adjustable in terms of its form or shape due to the layer structure, thereby allowing the release of the active ingredient and/or additive compound to be easily adjustable.

Preferably, the at least one layer, in particular only the at least one layer, forms a wall or is part of a wall of a pouch.

The composition may also be part of the wall of the pouch and/or be contained in a cavity surrounded by the wall of the pouch.

Preferably, at least two layers, in particular two layers, form the walls of the pouch, and the composition is surrounded by the at least two walls of the pouch.

Alternatively, at least two layers, in particular two layers, form the walls of the pouch, and the composition is part of the wall of the pouch.

Preferably, at least two layers, in particular two layers, form the walls of the pouch, and at least one of the at least two layers is coated (layered) and/or penetrated by the composition.

Advantageously the at least one layer of the oral product protects the user from coming into direct contact with the composition and in particular the active ingredient, that may be absorbed through the skin before the intended use as an oral product in the mouth.

The afore mentioned forms of the oral product have the advantage to exhaustively release the composition, the active ingredient and/or the additives, preferably after 1 min to 10 min, so that the oral product, preferably the pouch, can be removed from the mouth in adequate time.

According to a second aspect the invention relates to a method for preparing an oral product according to a first aspect.

The method comprises, in particular sequential in time, the steps of
a) providing at least one layer and
b) coating (layering) and/or penetrating, preferably uniformly coating (layering) and/or penetrating, the at least one layer with a composition.

The composition may be heated, in particular while stirring, to a temperature of 50° C. to 70° C., preferably 55° C. to 65° C., before carrying out step b).

Preferably, it depends on the temperature of the composition before and/or during carrying out step b), weather the at least one layer is coated (layered) or penetrated with the composition.

Furthermore, a temperature of the composition during step b) between 50° C. and 70° C. is preferred, when the at least one layer is penetrated by the composition.

And a temperature of the composition during step b) between 35° C. and 50° C. is preferred, when the at least one layer is coated (layered) by the composition.

Preferably, step b) is carried out by means of spraying, brushing, rolling or injection.

Preferably, the composition is liquid during step b), which simplifies a uniformly coating (layering) and/or penetrating of the composition.

The method may comprise a further step c) applying at least one further layer on the composition coating (layering) and/or the at least one layer being penetrated with the composition.

The method may comprise a further step bc) or step d) carrying out a reinforcing step, in particular embossing, stitching or needle felting.

Preferably, the at least one layer being coated and/or penetrated with the composition is cooled, in particular to a temperature of 15° C. to 50° C., preferably 20° C. to 30° C., before carrying out step bc) or d).

The method may comprise a further step e) carrying out a drying step. In other words, the oral product, in particular the composition is dried during step e). Preferably, the oral product comprising the composition may be free of water, in particular substantially free of water, after the oral product, in particular the composition, has been dried.

The method may comprise a further step c) or f) carrying out cutting, pre-perforating or stamping to form or shape the oral product.

Advantageously, due to the layer structure, the oral product is easily adjustable in terms of its form or shape by cutting and thereby allowing the release of the active ingredient to be easily adjustable.

With respect to further features and advantages of the method, reference is made in its entirety to the features and advantages described in terms of the oral product according to the first aspect of the invention. The features and advantages described under the first aspect of the invention do apply, mutatis mutandis, with respect to the method according to the second aspect of the invention.

Further features and advantages of the invention will become clear from the following examples in conjunction with the subject-matter of the dependent claim. The individual features can be realized either singularly or severally in combination in one embodiment of the invention. The preferred embodiments merely serve for illustration and better understanding of the invention and are not to be understood as in any way limiting the invention.

### BRIEF DESCRIPTION OF THE FIGURES

In the following, embodiments of the invention will be described in detail with reference to the drawings.

The figures schematically show the following:

FIG. 1: an embodiment of an oral product according to the invention before mechanical connection of the layers

FIG. 2: an embodiment of an oral product according to the invention as shown in FIG. 1 during mechanical connection of the layers

FIG. 3: an embodiment of an oral product according to the invention as shown in FIG. 1 and FIG. 2 after mechanical connection of the layers

### MORE DETAILED DESCRIPTION OF THE FIGURES

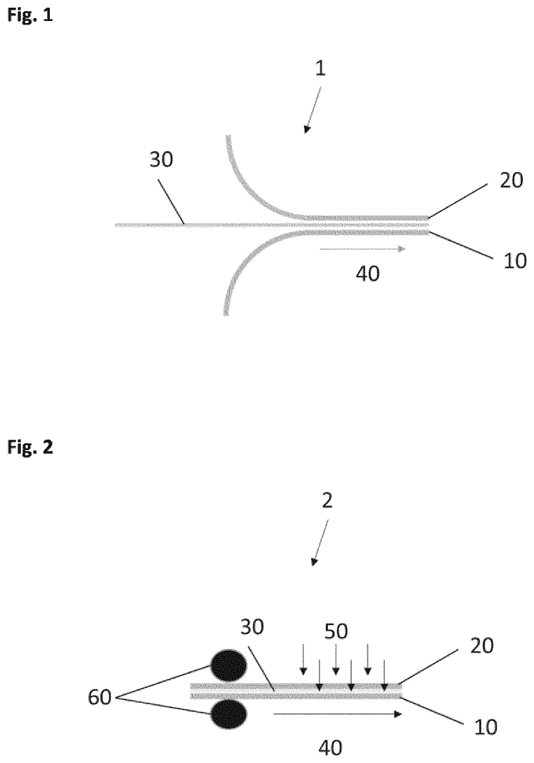

FIG. 1 schematically shows an embodiment of an oral product (1) for delivery of an active ingredient according to the invention before mechanical connection of the layers.

The oral product (1) in FIG. 1 comprises:
a first layer (10)
coated (layered) with a composition (composition layer) comprising a hydrogel forming component and the active ingredient (30),
a second layer (20), arranged above the first layer (10) coated with the composition comprising a hydrogel forming component and the active ingredient (30).

Alternatively, the composition layer (30) can be a third layer, preferably a third middle layer, which is coated (layered) and/or penetrated by the composition.

The first layer (10), the second layer (20) and the composition (30) are not yet connected in FIG. 1. However, on the right side of the figure the layers and the composition are firmly placed above each other, while on the left side of the figure the layers and the composition are not yet placed above each other.

Preferably, the second layer (20) is located above the inner composition (30), and the first layer (10) is located underneath the composition (30).

FIG. 2 schematically shows an embodiment of an oral product (2) for delivery of an active ingredient according to the invention before connection of the layers.

The oral product (2) in FIG. 2 comprises:
a first layer (10),
coated (layered) with a composition (composition layer) comprising a hydrogel forming component and the active ingredient (30),
a second layer (20), arranged above the first layer (10) coated with the composition comprising a hydrogel forming component and the active ingredient (30).

Alternatively, the composition layer (30) can be a third layer, preferably a third middle layer, which is coated (layered) and/or penetrated by the composition.

The first layer (10), the second layer (20) and the composition (30) are being connected to each other by means of needle felting. Wherein the arrows 50 indicate a needle being stabbed into the second layer (20) and the first layer (10), coated with the composition comprising a hydrogel forming component and the active ingredient (30), multiple times.

Further the first layer, the second layer and the composition are being connected to each other by means of embossing. Wherein two stamps (60) are put opposite of each other, one stamp on to the second layer (20) and one stamp onto the first layer (10), coated with the composition comprising a hydrogel forming component and the active ingredient (30), with a certain force to connect the layers.

The arrow 40 indicates the direction of production flow.

Preferably the layers (10, 20) are connected, in particular mechanically connected, to each other, preferably by means of embossing (imprinting) and/or needle felting.

Further preferred, the layers (10, 20) are in the form of textile layers.

Preferably, the textile layers are in the form of nonwoven fabric.

Preferably, the layers (10, 20) and the layer of composition (30) may be equal or different in terms of layer thickness.

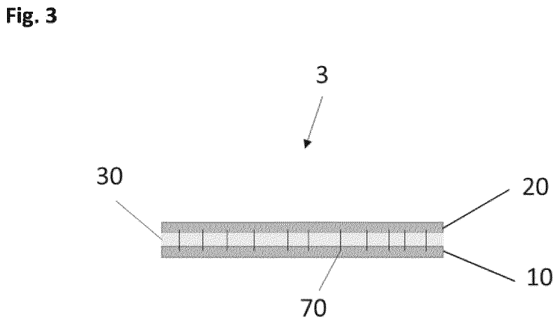

FIG. 3 schematically shows an embodiment of an oral product (3) for delivery of an active ingredient according to the invention before connection of the layers.

The oral product (3) in FIG. 3 comprises:
a first layer (10)
coated (layered) with a composition (composition layer) comprising a hydrogel forming component and the active ingredient (30),
a second layer (20) arranged above the first layer (10) coated with the composition comprising a hydrogel forming component and the active ingredient (30).

Alternatively, the composition layer (30) can be a third layer, preferably a third middle layer, which is coated (layered) and/or penetrated by the composition.

The first layer (10), the second layer (20) and the composition (30) are connected to each other by means of needle felting. Wherein the first layer (10) and the second layer (20) are entangled on itself, leading the first layer (10), the second layer (20) and the composition (30) to stick together making the connection dens and firm.

Preferably, the oral product comprising the composition may be free of water, in particular substantially free of water, after the oral product, in particular the composition, has been dried.

Preferably, the oral product (1,2,3) is formed into a pouch, sucking article such as bonbon, or a lozenge.

Further preferred, the first and second layer (10, 30) forms a wall or is part of a wall of a pouch, and the composition (30) is surrounded by the at least two walls of the pouch.

With respect to further features and advantages of the embodiments shown in the figures, reference is made in its entirety to the general description.

### EXPERIMENTAL SECTION

### Example 1

### Hydrogel Preparation:

Water, erythritol, sucralose, salt and carrageen, more specific kappa-carrageen, are heated under stirring to approx. 70° C. until the kappa-carrageen is dissolved.

Nicotine solved in glycerine and flavours are prewarmed and added. Stirring follows until a clear hydrogel solution is achieved.

### Preparation of the Oral Product:

While still warm and having an acceptable viscosity, the heated hydrogel solution is applied on a layer in a defined weight/area, e.g. equalling to a distinct amount of ingredient per final piece of product.

Depending on the temperature of the hydrogel, the hydrogel is either absorbed into or stays on the surface of the layer.

The coated layer is cooled to RT (room temperature ca. 22° C.-27° C.) or until the gel solidifies respectively. The layer with the solidified hydrogel is placed between two untreated layers of the same or different material and the layers are attached to each other by mechanical connection.

Formulation sequences, times and temperatures are dependent on the specific formulation.

Preferably, orals products were produced with a weight of 400 mg/oral product. Further preferred, each oral product comprises 6 mg nicotine, 40 mg caffeine and/or 10 mg CBD.

### Hydrogel Compositions:

Master composition 1: Erythritol (10%), sucralose (0.6%), sodium chloride (1%), 10% nicotine w/w in glycerine (15%), flavour Mint-Mix (2.5%) and water added to 100%.

Kappa-Carrageen was added to the master composition 1 in amounts of 0.5%, 1%, 1.5%, 2%, or 3%.

With increasing amount of kappa-carrageen the hydrogel went from a soft hydrogel due to low levels of hydrogel forming component to a hard gel. Even though all gels are suitable for the use in the inventive oral product, the ideal gel was achieved with 1.5% Kappa-Carrageen.

In one simplified composition no sucralose and no flavours were used and kappa-carrageen in an amount of 1.5% was added. The hydrogel was comparable to the above compositions with sucralose, flavours and 1.5% kappa-carrageen.

The compositions show that inventive hydrogels with different kappa-carrageen concentrations tested in appearance, pH, hardening properties, temperature needed for liquefaction and temperature needed for solidification, show suitable results for a use in the inventive oral product. However, compositions with 1% to 2% kappa-carrageen, in particular composition with 1.5% kappa-carrageen showed the best suitability for the inventive oral product in terms of all components being soluble at max 60° C., the warm gel solution having a viscosity which guarantees easy processing and pumpability, the gel setting being reversible at <40° C. and a pH of the gel solution being adjustable to pH 7.5-8.5, while the formed hydrogel can dry and release water, in particular most or all water.

### Example 2

### Hydrogel Preparation:

Water, erythritol, salt and a hydrogel forming component (GFA) in the form of gelatine, agar, polyvinyl pyrrolidone (PVP 25/PVP90) or alginate are heated under stirring to approx. 70° C. until the GFA is dissolved.

When gelatine was used, the solution was heated to 35° C. until the gelatine was dissolved, when agar was used the solution was heated until boiling temperature of 90° C. to 95° C. to dissolve the agar and when PCP25/PVP90 was used the solution was only gently heated to dissolve the PVP25/PVP90.

Nicotine in glycerine and flavours are prewarmed and added. Stirring follows until a clear hydrogel solution is achieved.

Preparation of the oral product is performed as described under example 1.

### Hydrogel Compositions:

Master composition 2a: Erythritol (10%), sodium chloride (1%), 10% nicotine w/w in glycerine (15%), flavour Mint-Mix (2.5%) and water added to 100%.

Gelatine was added to the master composition 2a in amounts of 2% or 3%. The pH of the compositions was 8.78 and 8.72 respectively.

With increasing amount of gelatine, the hydrogel went from a soft hydrogel due to low levels of hydrogel forming component to a hard hydrogel. Even though all gels are suitable for the use in the inventive oral product, the ideal gel was achieved with 3% gelatine.

Master Composition 2b: Erythritol (10%), Sodium Chloride (1%), 10% Nicotine w/w in Glycerine (15%) and water added to 100%.

Either agar-agar was added to the master composition 2b in amounts of 0.8% or 1.2%. The pH of the compositions was 8.3 and 8.2 respectively, or alginate was added to the master composition 2b in amounts of 1.5% or 2.25%, or PVP25 (Kollidon25®) or PVP90 (Kollidon90®) was added to the master composition 2b in amounts of 5%. The pH of the compositions was 8.1 and 7.9 respectively.

With increasing amount of agar-agar, the hydrogel went from a soft hydrogel due to low levels of hydrogel forming component to a hard hydrogel. Even though all hydrogels are suitable for the use in the inventive oral product, the ideal gel was achieved with 3% agar-agar. Compositions with PVP25 (Kollidon25®) or PVP90 (Kollidon90®) or alginate all showed suitable hydrogels for the use in the inventive oral product.

The compositions show the suitability of the hydrogels with gelatine, agar-agar, PVP or alginate as GFA for the use in the inventive oral product.

The hydrogels were tested in appearance, pH, hardening properties, temperature needed for liquefaction and temperature needed for solidification.

The compositions showed suitable results for a use in the inventive oral product. In particular in terms of all components being soluble, the warm gel solution having a viscosity which guarantees easy processing and pumpability and a pH of the gel solution adjustable to pH 7.5-8.5, while the formed hydrogel can dry and release water, in particular most or all water.

### Example 3

### Hydrogel Preparation:

Water, erythritol, salt, pH regulator and a hydrogel forming component (GFA) in the form of carrageen, in particular kappa carrageen, are heated under stirring to approx. 70° C., in particular 65° C., until the GFA is dissolved.

All ingredients were weighted together, and solids were added first then liquids.

Nicotine in glycerine and flavours are prewarmed and added. Stirring follows until a clear hydrogel solution is achieved.

Preparation of the oral product is performed as described under example 1.

### Hydrogel Compositions:

Master composition 3: Erythritol (10%), sodium chloride (1%), 10% nicotine w/w in glycerine (15%), kappa-carrageen (1.5%) and water added to 100%.

Citric acid, NaOH or Na₂CO₃ was added to the master composition 3 in an amount of 2%.

All gels are suitable for the use in the inventive oral product and ideal gels were achieved.

The compositions show the suitability of the hydrogels with different pH regulators (citric acid, NaOH, Na₂CO₃) for the use in the inventive oral product. The hydrogels were tested in appearance, pH, hardening properties, temperature needed for liquefaction and temperature needed for solidification. The compositions showed suitable results for a use in the inventive oral product. In particular in terms of all components being soluble at max. 60° C., the warm gel solution having a viscosity which guarantees easy processing and pumpability, the gel setting being reversible at <40° C. and a pH of the gel solution being adjustable to pH 7.5-8.5 without compromising the gel formation, while the formed hydrogel can dry and release water, in particular most or all water.

### Example 4

### Hydrogel Preparation:

Water, polyols, salt and a hydrogel forming component (GFA) in the form of carrageen, in particular kappa-carrageen, are heated under stirring to approx. 70° C., in particular 65° C., until the GFA is dissolved.

When mannitol was used, the solution was heated to 62° C. until a clear solution was achieved, when maltitol was used the solution was heated to 65° C. until a clear solution was achieved and when xylitol was used the solution was heated to 52° C. until a clear solution was achieved.

Further, all ingredients were weighted together, solids were added first then liquids.

Nicotine in glycerine and flavours are prewarmed and added. Stirring follows until a clear hydrogel solution is achieved.

Preparation of the oral product is performed as described under example 1.

### Hydrogel Compositions:

Master composition 4: Sodium chloride (1%), 10% nicotine w/w in glycerine (15%), kappa-carrageen (1.5%) and water added to 100%.

Mannitol, maltitol or xylitol was added to the master composition 4 in an amount of 2%.

All gels are suitable for the use in the inventive oral product and ideal gels were achieved.

The compositions show the suitability of the hydrogels with different polyols (mannitol, maltitol, xylitol) for the use in the inventive oral product.

The hydrogels were tested in appearance, pH, hardening properties, temperature needed for liquefaction and temperature needed for solidification.

The compositions showed suitable results for a use in the inventive oral product. In particular in terms of all components being soluble at max. 60° C., the warm gel solution having a viscosity which guarantees easy processing and pumpability, the gel setting being reversible at 45° C., in particular a full liquefaction occurred at 65° C., and the hydrogel hardened again as before without colour change while the polyols did not compromise the hydrogel formation, and a pH of the gel solution being adjustable to pH 7.5-8.5, while the formed hydrogel can dry and release water, in particular most or all water.

### Example 5

### Hydrogel Preparation:

Water, erythritol, salt and a hydrogel forming component (GFA) in the form of carrageen, in particular kappa-carrageen, are heated under stirring to approx. 70° C., in particular 65° C., until the GFA is dissolved.

Active ingredients (caffeine or CBD (cannabidiol)) are prewarmed and added. Stirring follows until a clear hydrogel solution is achieved.

After the active ingredient was added, the solution was preferably heated to 75° C. until a clear solution was achieved, when CBD was used, the solution was heated to 65° C.

Water-soluble active ingredients like caffeine are especially good dissolved in the hydrogel. For lipophile active ingredients such as CBD a cloudy hydrogel is preferably formed to achieve homogenous distribution of the active ingredient in the hydrogel.

Preparation of the oral product is performed as described under example 1.

### Hydrogel Compositions:

Master composition 5: Erythritol (10%), sodium chloride (1%), kappa-carrageen (1.5%) and water added to 100%.

Pure caffeine or pure CBD was added to the master composition 5 in an amount of 10% or 2.5% respectively.

All hydrgels are suitable for the use in the inventive oral product and ideal hydrogels were achieved.

The compositions show the suitability of the hydrogels with active ingredients (caffeine or CBD (cannabidiol)) for the use in the inventive oral product.

The hydrogels were tested in appearance, pH, hardening properties, temperature needed for liquefaction and temperature needed for solidification. The compositions showed suitable results for a use in the inventive oral product. In particular in terms of all components being soluble at max 60° C., the warm gel solution having a viscosity which guarantees easy processing and pumpability, the gel setting being reversible at <40° C. and a pH of the gel solution being adjustable to pH 7.5-8.5, while the formed hydrogel can dry and release water, in particular most or all water.

### Example 6

### Hydrogel Preparation:

Water, erythritol, salt and a hydrogel forming component (GFA) in the form of carrageen, in particular kappa-carrageen, are heated under stirring to approx. 70° C., in particular 65° C., until the GFA is dissolved.

Nicotine in glycerine and flavours are prewarmed and added. Stirring follows until a clear hydrogel solution is achieved.

Preparation of the oral product is performed as described under example 1.

### Hydrogel Compositions:

Master composition 6 A-D: Erythritol (A-D: 10 mg), sodium chloride (A-D: 1 mg), 10% nicotine w/w in glycerine (A-D: 15 mg), kappa-carrageen (A: 1.5 mg, B: 0.5 mg, C: 3 mg, D: 3 mg), Water (A: 22.5 mg, B: 23.5 mg, C: 121 mg, D: 171 mg), total weight: (A: 50 mg, B: 50 mg, C: 150 mg, D: 200 mg).

All hydrogels are suitable for the use in the inventive oral product and ideal hydrogels were achieved.

The compositions show the suitability of the hydrogels with different water concentrations.

The hydrogels were tested in appearance, pH, hardening properties, temperature needed for liquefaction and temperature needed for solidification. The compositions showed suitable results for a use in the inventive oral product. In particular in terms of all components being soluble at max 60° C., the warm gel solution having a viscosity which guarantees easy processing and pumpability, the gel setting being reversible at <40° C. and a pH of the gel solution being adjustable to pH 7.5-8.5, while the formed hydrogel can dry and release water, in particular most or all water.

The hydrogels were tested in appearance, pH, hardening properties, temperature needed for liquefaction and temperature needed for solidification.

The compositions showed suitable results for a use in the inventive oral product. In particular in terms of all components being soluble at max 60° C., the warm gel solution having a viscosity which guarantees easy processing and pumpability, the gel setting being reversible at <40° C. and a pH of the gel solution being adjustable to pH 7.5-8.5, while the formed hydrogel can dry and release water, in particular most or all water.

1. An oral product for delivery of an active ingredient comprising
at least one layer consisting of water-insoluble fibres and
a composition comprising a hydrogel forming component and the active ingredient, wherein the oral product is capable of releasing the active ingredient without the need of chewing the oral product, the at least one layer is in the form of at least one textile layer and the at least one layer is penetrated by the composition, the composition is in the form of a hydrogel, the active ingredient is selected from the group consisting of nicotine, caffeine, taurine, cannabinoids, cannabinol, cannabidiol, cannabigerol, tetrahydrocannabinol, melatonin, theobromine, and any combination thereof, and the composition forms an inner composition layer within the at least one layer.

2. The oral product according to claim 1, wherein the at least one layer is in the form of only one layer or in the form of a plurality of layers, in particular 2 to 5 layers, wherein the layers are arranged one above the other.

3. The oral product according to claim 2, wherein at least one inner layer, in particular only one inner layer, of the plurality of layers is coated and/or layered and/or penetrated by the composition.

4. The oral product according to claim 2, wherein the layers are connected, in particular mechanically connected, to each other.

5. The oral product according to claim 1, wherein the at least one textile layer is in the form of at least one nonwoven fabric, at least one felt fabric, at least one knitted fabric or at least one woven fabric.

6. The oral product according to claim 1, wherein the at least one layer comprises water-insoluble natural fibres, and/or synthetic fibres.

7-8. (canceled)

9. The oral product according to claim 1, wherein the hydrogel forming component is selected from the group consisting of carrageen, agar-agar, alginates, polyvinyl pyrrolidone, polyvinyl acetate, cellulose derivative, and mixtures of at least two of the afore-said hydrogel forming components.

10. The oral product according to claim 1, wherein the active ingredient is in a free form, i.e. not ionic and/or bound form, in an ionic form, i.e. in the form of a salt, or in a bound, in particular complexed, form.

11. (canceled)

12. The oral product according to claim 1, wherein the active ingredient is released from the composition or oral product during a period of 0.5 min to 60 min, in particular 1 min to 30 min, preferably 1 min to 10 min.

13. The oral product according to claim 1, wherein the oral product, in particular the composition, comprises at least one additive compound, preferably selected from the group consisting of pH regulator, flavour, sugar substitute, sweetener, polyol, humectant, colour, preservative and mixtures of at least two of the afore-said additive compounds.

14. The oral product according to claim 1, wherein the oral product is in the form of a pouch, sucking article such as bonbon, or a lozenge.

15. The oral product according to claim 2, wherein the layers are connected with each other by means of stitching, embossing or needle felting.

16. The oral product according to claim 1, wherein the at least one layer comprises water-insoluble plant fibres and/or water-insoluble animal fibres.

17. The oral product according to claim 1, wherein the at least one layer comprises fibres selected from the group consisting of cotton fabric, silk fabric, linen fabric, wool fabric, lycra fabric, viscose fabric, nylon fabric, polyester fabric, rayon fabric, polyethylenterephthalat (PET) fabric and combinations of at least two of the afore-said fibres.

18. The oral product according to claim 9, wherein the cellulose derivative is carboxy methylcellulose, hydroxypropyl methylcellulose, or a combination thereof.
